(19) United States
(12) Patent Application Publication
(10) Pub. No.: US 2011/0214315 A1
(43) Pub. Date: Sep. 8, 2011

(54) THERAPY SHOE

(75) Inventors: Matthew Mayer, Grand Junction, CO (US); David M. Mayer, Grand Junction, CO (US); Gerhard Rill, Parachute, CO (US)

(73) Assignee: LEAP FROGG, LLC, Grand Junction, CO (US)

(21) Appl. No.: 13/040,982

(22) Filed: Mar. 4, 2011

Related U.S. Application Data
(60) Provisional application No. 61/311,018, filed on Mar. 5, 2010.

(51) Int. Cl. A61F 5/14

(52) U.S. Cl. 36/140

(57) ABSTRACT

Methods and systems for aiding with dynamic compression of venous tissue enable improved blood movement, reducing edema, reducing leg cramping, treating plantar fasciitis in the extremities, and/or the like. In an exemplary embodiment, a therapy shoe entirely contains a therapy device which provides a compressive force to the venous plexus region of the foot. The system may assist blood circulation which may reduce the occurrence of undesirable complications such as deep vein thrombosis, ulcers, and the like.

### CROSS-REFERENCE TO RELATED APPLICATIONS

This application is a non-provisional of U.S. Provisional No. 61/311,018 filed on Mar. 5, 2010 and entitled “THERAPY SHOE”, wherein such provisional application is hereby incorporated in its entirety, by reference.

### TECHNICAL FIELD

The present disclosure generally relates to systems and methods for providing medical therapy and specifically to systems and methods for assisting the stimulation of blood flow, reducing edema, reducing leg cramping, treating plantar fasciitis in the foot, and/or promoting recovery from athletic excursion. More specifically, the present disclosure generally relates to a shoe configured to hold a removable and/or integrated device that applies pressure to a wearer's foot to treat various ailments and/or provide athletic recovery after physical exercise.

### BACKGROUND

In order to enhance circulation in a person's body, particularly in the feet and legs, periodic or cyclic compression of tissue, such as plexus regions of the foot, at predetermined timed intervals is beneficial. Under normal circumstances, blood moves up the legs due to muscle contraction and general movement of the feet or legs, such as when walking. If a person is immobilized, unable to move regularly, or has poor circulation brought on by disease, the natural blood return mechanism is impaired, and circulatory problems such as ulcers and deep vein thrombosis can occur.

To mitigate these problems, it is desirable to concentrate a compression force against veins throughout the legs and/or feet. Current systems are primarily based on pneumatic compression devices that squeeze the entire foot, calf, or thigh. These systems require significant power, and are inefficient because they provide high levels of force across the entire foot or leg rather than focusing in on those areas with the highest concentration of blood vessels. In addition, these systems may include air bags that can rupture at the seam, especially with high pressure within the bag.

In various current devices, tethered air lines limit mobility, and can lead to injury should the person attempt to walk while the device is in use. Further, existing devices may not be suited for continuous usage. Users cannot walk with them, or move away from the compression unit. The device must be removed before a user can walk. Additionally, current devices lack the ability to track and report user usage and compliance. Also, most pneumatic devices are quite noisy and can cause irritation of the skin leading to ulcers.

Furthermore, current devices do not blend in well with ordinary clothing and stand out as medical devices. It would be advantageous to provide a therapy shoe that could be worn as ordinary clothing and not be an obvious medical aid.

### SUMMARY

In an exemplary embodiment, an item of footwear comprises a housing, a therapy device disposed within a cavity in the housing, and a coupling portion. The coupling portion is configured to retain a portion of a foot.

In another exemplary embodiment, a method for compressing a foot comprises providing an item of footwear having a therapy device entirely contained therein, coupling the item of footwear to the foot, and activating the therapy device.

In another exemplary embodiment, an athletic shoe comprises a housing configured with an aperture having a flexible aperture cover. The athletic shoe further comprises a foot compression system disposed within the housing. The foot compression system comprises a pressure pad extendable to at least partially displace the flexible aperture cover into contact with the venous plexus region of a foot. The housing is configured with an opening on an external side of the housing. The opening allows access to a control component of the foot compression system.

In another exemplary embodiment, an item of footwear comprises a housing having an aperture therethrough. The aperture is configured with a flexible aperture cover. The item of footwear further comprises a sole, and a therapy device disposed between the housing and the sole. The therapy device is configured to extend at least partially through the aperture to compress a portion of a foot. The item is footwear is configured to fully conceal the presence of the therapy device when the item of footwear is worn.

The contents of this summary section are provided only as a simplified introduction to the disclosure, and are not intended to be used to limit the scope of the appended claims.

### BRIEF DESCRIPTION OF THE DRAWINGS

With reference to the following description and accompanying drawings:

FIG. 1 illustrates a therapy shoe in accordance with an exemplary embodiment;

FIG. 2 illustrates a perspective view of the therapy shoe of FIG. 1;

FIG. 3 illustrates a top view of the therapy shoe of FIGS. 1 and 2;

FIG. 4 illustrates a foot compression system coupled to a therapy shoe in accordance with an exemplary embodiment;

FIG. 5 is a block diagram of a foot compression system coupled to a therapy shoe in accordance with an exemplary embodiment; and

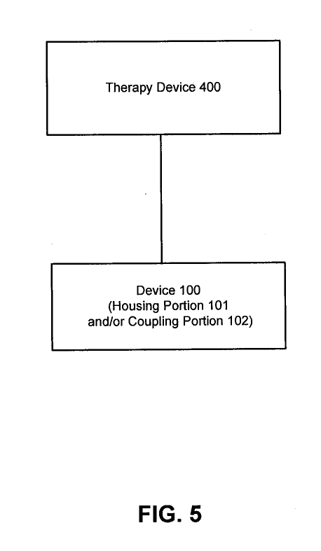

FIG. 6 illustrates a therapy shoe coupled to a compression garment in accordance with an exemplary embodiment.

### DETAILED DESCRIPTION

Details of the present disclosure may be described herein in terms of various components and processing steps. It should be appreciated that such components and steps may be realized by any number of hardware and/or software components configured to perform the specified functions. For example, a therapy device configured to aid a portion of a lower extremity and/or foot compression system, or a system for athletic recovery, may employ various medical treatment devices, input and/or output elements and the like, which may carry out a variety of functions under the control of one or more control systems or other control devices. In addition, principles of the present disclosure may be practiced in any number of medical or treatment contexts, and exemplary embodiments relating to a deep vein thrombosis treatment system as described herein are merely a few of the exemplary applications. For example, principles of the present disclosure may be utilized in connection with athletic recovery. Yet further, the principles, features and methods discussed may be applied to any suitable medical or other tissue or treatment application.

Moreover, principles of the present disclosure may suitably be combined with principles of foot compression systems and methods disclosed in U.S. patent application Ser. No. 12/499,473 filed on Jul. 8, 2009, now published as U.S. Patent Application Publication No. 2010/0010398 entitled “FOOT COMPRESSION SYSTEM”, the contents of which are hereby incorporated by reference in their entirety. Principles of the present disclosure may also suitably be combined with principles of foot compression systems and methods disclosed in U.S. patent application Ser. No. 13/004,754 filed on Jan. 11, 2011 entitled “FOOT COMPRESSION SYSTEM”, the contents of which are hereby incorporated by reference in their entirety.

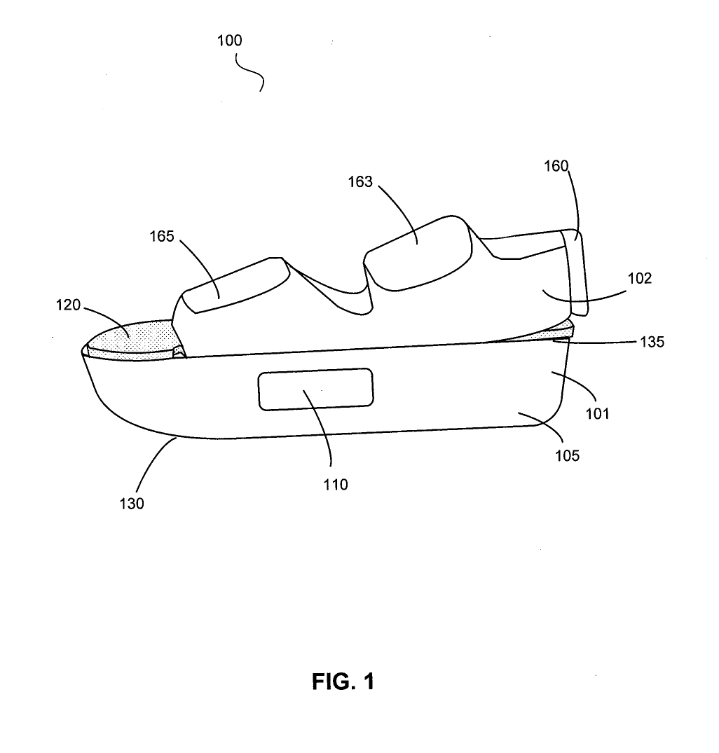

An item of therapeutic footwear, for example therapy shoe 100, may be any device configured to aid in delivering a compressive force to a portion of a wearer, for example a human foot. With reference now to FIGS. 1-3, and in accordance with an exemplary embodiment, therapy shoe 100 comprises housing portion 101 and coupling portion 102.

With further reference now to FIG. 1, and in accordance with an exemplary embodiment, therapy shoe 100 may comprise housing portion 101 and coupling portion 102. In one exemplary embodiment, housing portion 101 retains a therapy device, such as therapy device 400 and/or other therapy devices capable of providing medical therapy to a portion of a wearer, such as a surface of the foot. In various exemplary embodiments, therapy device 400 may comprise a foot compression system, for example as disclosed in U.S. patent application Ser. No. 13/004,754 filed on Jan. 11, 2011 entitled “FOOT COMPRESSION SYSTEM”. In one exemplary embodiment, a therapy device, such as therapy device 400, is integral to therapy shoe 100. In one exemplary embodiment, a therapy device, such as therapy device 400, is removably coupled to therapy shoe 100.

In various exemplary embodiments, therapy shoe 100 may be configured for use in various medical applications, for example increasing blood flow, treating and/or preventing deep vein thrombosis, lower leg edema, ulcers, and/or the like. Moreover, therapy shoe 100 may be configured for use in athletic recovery, for example in accordance with principles of athletic recovery as disclosed in U.S. patent application Ser. No. 13/004,754 filed on Jan. 11, 2011 entitled “FOOT COMPRESSION SYSTEM”.

Although one exemplary embodiment of therapy shoe 100 is described and shown as a sandal-like item of footwear, principles of the present disclosure is not limited to therapy shoe 100 as explicitly shown. Specifically and without limitation, therapy shoe 100 can be any piece of footwear or clothing that is configured to at least partially contain, fully contain, and/or couple to therapy device 400. In that regard, therapy shoe 100 can be (but is not necessarily limited to) in the form of a sock, slipper, sandal, tennis shoe, running shoe, athletic shoe, boot, dress shoe, high heel shoe, mule, slingback, clog, etc. Further, therapy shoe 100 can be in produced in various design(s) to appeal to various fashion tastes. In various exemplary embodiments, therapy shoe 100 is designed to look similar to another piece of footwear and/or be difficult to distinguish from (or even indistinguishable from) conventional footwear lacking functionality of therapy shoe 100.

In one exemplary embodiment, coupling portion 102, coupled to housing portion 101, secures a therapy device, such as therapy device 400, to a portion of a wearer, such as a surface of the foot like the venous plexus region. Housing portion 101 and coupling portion 102 may be made out of any suitable materials and/or fashioned in any suitable architecture and/or geometry, to perform their respective functions.

In one exemplary embodiment and with renewed reference to FIG. 1, housing portion 101 is located in and/or integral to the base of a wearable therapy shoe 100. In an exemplary embodiment, housing portion 101 may be one of enclosed, partially enclosed or exposed. In one exemplary embodiment, housing portion 101 may comprise a housing cavity (not explicitly shown in FIG. 1) defined by a top housing top surface 135, a housing bottom surface 130 and at least one housing side surface 105. In this embodiment, the dimensions of the housing cavity may be designed to hold a particular device, for example therapy device 400, which may be housed by housing portion 101. For example, in one exemplary embodiment, the housing cavity may be designed to be slightly larger than a provided therapy device 400. Thus, the housing cavity may effectively mirror the exterior dimensions of therapy device 400 housed by the housing cavity. Alternatively, in one exemplary embodiment, the housing cavity may be designed to mirror the exterior dimensions of therapy device 400 housed in the housing cavity with additional cavity portions configured to receive and/or retain additional elements. These additional elements may include power supplies, straps to retain a therapy device, spacers, counter balances, shock absorbers, cushioning, sensors, structural supports, and/or the like.

In one exemplary embodiment, housing side surface 105 may comprise one contiguous surface that effectively wraps around and/or partially encloses housing portion 101. In another exemplary embodiment, housing side surface 105 may comprise a plurality of surfaces that combine to form a portion of housing portion 101. Housing side surface 105 may be made from any suitable material for providing effective containment and/or retention of a therapy device, such as therapy device 400. In another exemplary embodiment, housing side surface 105 may be made from any suitable material for providing effective structural support to support the weight of a user. For example, in one exemplary embodiment, housing side surface 105 is made from one or more of plastic, foam, metal, cork, wood, natural rubber, synthetic rubber, composite, or other durable material. Housing side surface 105 may be a constant width or may comprise a variable width. In one exemplary embodiment, the width of housing side surface 105 is a function of the height of therapy device 400. In one exemplary embodiment, the width of housing side surface 105, (i.e. the height of the housing portion 101) is as small as possible. In one exemplary embodiment, the heel area of therapy shoe 100 may comprise additional heel support. This heel support may be structural and/or configured to provide shock absorption.

In one exemplary embodiment, a surface, such as housing side surface 105, housing top surface 135, and/or housing bottom surface 130, comprises an opening 110 to access a portion of a provided therapy device, such as therapy device 400. In one exemplary embodiment, with renewed reference to FIG. 1, opening 110 is located on housing side surface 105. In one exemplary embodiment, opening 110 may comprise any suitable dimensions for accessing a provided therapy device, such as therapy device 400. This accessing may be visual and/or physical. For instance, the dimensions and/or location of opening 110 may be related to the positioning of controls and/or indicators on a provided therapy device, such as therapy device 400. Such controls and/or indicators may comprise on/off buttons, lights, switches, and/or the like. Access to the same may be provided via opening 110.

In one exemplary embodiment, a cover (not shown in the figures) may be coupled to opening 110 to prevent debris and/or unwanted matter from entering housing portion 101. In one exemplary embodiment, the cover may be transparent. The cover may be coupled to opening 110 via any suitable components and/or methods, such as pressure fit, latch, screw, glue, epoxy, snap, and/or the like. Further, the cover may made from any suitable material, such as the same material as housing side surface 105, or a material other than the same material as housing side surface 105. For instance, the cover material may be at least partially transparent and/or flexible, for example in order to allow a user to manipulate the controls of therapy device 400, while still preventing debris from entering the housing portion 101.

In various exemplary embodiments, therapy shoe 100 may be configured absent opening 110. In these exemplary embodiments, access to controls of therapy device 400 may be provided via one or more wires passing through housing portion 101, via wireless communication with therapy device 400, and/or via any other suitable method, as desired. Moreover, in certain exemplary embodiments therapy device 400 may be configured to be powered and/or charged via an induction coupling.

In an exemplary embodiment, a gateway (not shown in the figures) is located on housing portion 101, for example coupled to housing bottom surface 130. In an exemplary embodiment, the gateway comprises any suitable dimensions for accessing, replacing, installing and/or removing a provided therapy device, such as therapy device 400. For instance, the dimensions of the gateway may be a function of the geometry of the exterior (housing) of therapy device 400. In one exemplary embodiment, the access panel may be coupled to the gateway to prevent debris and/or unwanted matter from entering housing portion 101 and/or to prevent a provided therapy device 400 from decoupling from housing portion 101. The access panel may be coupled to the gateway via any suitable components and/or methods, such as pressure fit, latch, screw, glue, epoxy, snap, and/or the like. An access panel (not shown) may be made from any suitable material, such as the same material as housing bottom surface 130 or a material other than the same material as housing bottom surface 130. For instance, in one embodiment, the access panel may be made from the same material and have the same surface texture as the outsole material.

In an exemplary embodiment, the exterior of housing bottom surface 130 (the outsole material) may be textured. For instance, the exterior of housing bottom surface 130 may be configured to comprise a tread pattern. In an exemplary embodiment, the texture on housing bottom surface 130 may be configured to improve traction of therapy shoe 100. Moreover, the texture on housing bottom surface 130 may be configured in any suitable pattern or configuration.

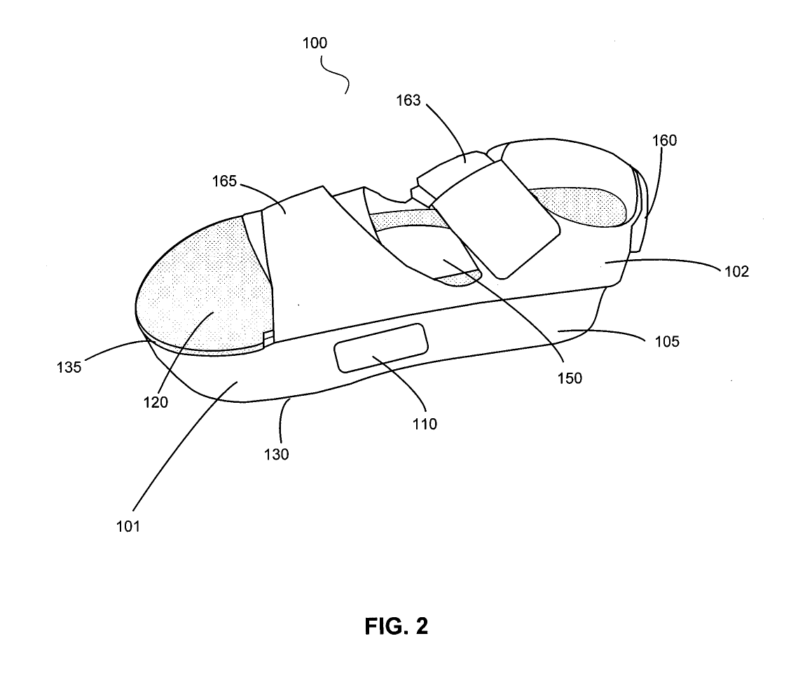

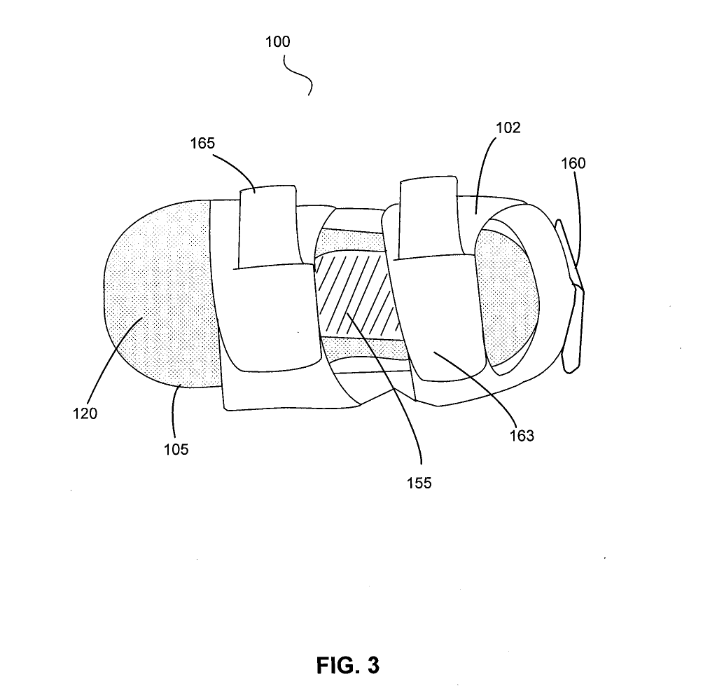

In an exemplary embodiment, with reference to FIGS. 2 and 3, aperture 150 is located on housing top surface 135. Aperture 150 may extend from housing portion 101 to coupling portion 102. In one exemplary embodiment, aperture 150 may comprise any suitable dimensions to assist therapy device 400 in providing therapy. For instance, the dimensions of aperture 150 may be a function of the orientation and geometry of therapy elements of a provided therapy device 400. In various exemplary embodiments, aperture 150 comprises an opening having an area from about 6 square centimeters to about 40 square centimeters. In various exemplary embodiments, aperture 150 comprises an opening configured to facilitate a provided therapy device, such as therapy device 400, making contact with the venous plexus region of the foot. For instance, aperture 150 may comprise an opening configured to facilitate therapy device 400 applying a compressive force to the arch region of a foot.

In certain exemplary embodiments, housing top surface 135 may be configured to be continuous (i.e., absent aperture 150). In these exemplary embodiments, at least a portion of housing top surface 135 may be configured to be flexible and/or deformable, for example in order to deform responsive to a force exerted by therapy device 400 and make contact with the venous plexus region of the foot.

In an exemplary embodiment, an aperture cover 155 may at least partially couple to, obstruct, and/or otherwise cover aperture 150 to prevent debris and/or unwanted matter from entering housing portion 101 while still facilitating contact of therapy device 400 with a foot. In general, aperture cover 155 is configured to be flexible and/or deformable.

For instance, in an exemplary embodiment aperture cover 155 may stretch from a first position and/or configuration where a force is not being provided by therapy device 400 to a portion of a wearer, to a second position and/or configuration where a force is being provided by therapy device 400 to a wearer. Likewise, in an exemplary embodiment, aperture cover 155 may return from a second position and/or configuration where a force is being provided by therapy device 400 to a portion of a wearer, to a first position and/or configuration where a force is not being provided by therapy device 400 to a wearer. Aperture cover 155 may be made from any suitable material configured to withstand providing force to a portion of a wearer. For instance, aperture cover 155 may be made from latex, plastic, rubber, synthetic, cloth, fabric, and or the like. In one exemplary embodiment, aperture cover 155 may be washable.

In various exemplary embodiments, aperture cover 155 comprises material configured to disperse pressure across a portion of a wearer. For instance, in one exemplary embodiment, aperture cover 155 comprises a non-solid material, such as a gel pack. This non-solid material may assist in the prevention of skin breakdown. In another exemplary embodiment, a soft material, such a cushioning, cotton, padding, foam, gel pack, gauze, and/or the like, may be coupled to and/or integral to aperture cover 155. In various exemplary embodiments, this soft material coupled to aperture cover 155 may aid in preventing skin breakdown. Moreover, aperture cover 155 may be coupled to aperture 150 via any suitable components and/or methods, such as pressure fit, latch, screw, glue, epoxy, snap, and/or the like.

In an exemplary embodiment, coupling portion 102 comprises surface 120. In one embodiment, surface 120 is coupled to the exterior surface of housing top surface 135. Surface 120 may be configured to at least partially receive and/or support a portion of a wearer, such as the sole of a foot. Accordingly, surface 120 may be configured with any suitable shape or dimension. In an exemplary embodiment, surface 120 is larger than a provided foot. Moreover, surface 120 may be made from any suitable material, such as foam, leather, sponge, rubber, plastic, synthetic, cork and/or the like.

In various exemplary embodiments, surface 120 is configured to provide a comfortable surface for touching a foot. For instance, surface 120 may comprise an insole configured to provide shock absorption. In various embodiments, the insole coupled to surface 120 may be removable, such as for washing and/or cleaning. Moreover, in various embodiments, custom orthotics may be integral to and/or coupled to surface 120.

In an exemplary embodiment, coupling portion 102 comprises couplers to receive and retain a portion of a wearer, such as a foot, ankle and/or portion of a lower extremity. The couplers may be custom sized material, belts, elastics, buckles, snaps, ties, hook and loop style fasteners (e.g., VELCRO® brand fasteners provided by Velcro USA, Inc.), buttons, zipper, sheaths, and/or the like.

In an exemplary embodiment, the couplers comprise adjustable straps, such as straps 160, 163, 165, configured to receive and retain a portion of a foot, such as dorsal surface, the heel, and/or the like, as is known. Moreover, therapy shoe 100 may comprise any suitable number of straps. In one exemplary embodiment, each strap 160, 163, 165 comprises a first side and a second side. The first side and/or second side are configured to comprise a portion of a fastener, such as a first hook and loop fastener portion and/or a second hook and loop fastener portion configured to mate with and couple to an opposite hook and loop fastener portion, on at least one of a top surface and/or or bottom surface of the first side and/or second side.

In an exemplary embodiment, with regard to strap 160, 163, 165, the first side is configured to have an opening which a portion of the second side may be fed through. A portion of second side of strap 160, 163, 165 is fed though an opening in a first side of strap 160, 163, 165 and is adjustably anchored using a fastener, such as a hook and loop fastener to a portion of first side of strap 160, 163, 165. In this embodiment, a portion of first side of strap 160, 163, 165 overlaps second side of strap 160, 163, 165 and is adjustably anchored using a fastener, such as a hook and loop fastener.

Straps (e.g. strap 160, 163, 165) may be located in any suitable location. In one embodiment strap 160 may located to receive and retain the rear portion of an ankle. In one embodiment strap 163 may located to receive and retain the front portion of an ankle and/a top surface of the foot. In one embodiment strap 165 may located to receive and retain a top surface of the foot. In another exemplary embodiment, a soft material, such a cushioning, cotton, padding, foam, gel pack, gauze, and/or the like, may be coupled to the inside surface of strap 160, 163, 165, to provide comfort for a user. Moreover, straps (e.g. strap 160, 163, 165) may be configured to accept variances in individual feet (e.g., height of arch, curvature of arch, width, length, and/or the like).

In other exemplary embodiments, straps (e.g. strap 160, 163, 165) are eliminated and other known footwear constructions are provided. These examples include, but are not limited to, laces, buttons, tongues, designer straps (such as those on a high heel shoe), and other similar constructions. Moreover, any material now known or used in the future to construct footwear may suitably be used, and is considered to fall within the scope of the present disclosure.

In an exemplary embodiment, a covering, such as a waterproof covering, may be coupled to therapy shoe 100. For example, a bag or shroud may be coupled around therapy shoe 100 to prevent unwanted elements such as sand, water, dirt, debris, and/or particulate from coming into contact with therapy shoe 100. In various exemplary embodiments, therapy shoe 100 may at least partially comprise natural rubber, synthetic rubber, polymer composites, or other waterproof and/or water-resistant materials.

In various exemplary embodiments, therapy shoe 100 is coupled to an external object. For instance, the external object may be a fixed surface, such as a wedge in a fixed position coupled to the ground. The external object may be an elevated surface, such as a footrest. Alternatively, the external object may be coupled to and/or comprise a movable object such as a portion of a bed, chair, hospital bed, cart, or wheelchair. The coupling may be by any suitable means, such as magnetic, mechanical, hook and loop fastener, strapping, fastening, lacing, tying, and/or the like.

In accordance with an exemplary embodiment, therapy shoe 100 is configured to be fabricated integral to a shoe, boot, sandal, or other footwear. Moreover, therapy shoe 100 may be configured with various sizes, including industry standard sizes. For instance, therapy shoe 100 may be formed in a size compatible to and related to a foot size such as at least one of: USA men's sizes: 6 through 16 (in any suitable width such as C, D, E, and/or the like); USA women's sizes: 4 through 14 (in any suitable width such as narrow, average, wide, extra wide, and/or the like); USA children's sizes: 6 through 13 and 1 through 3; European men's sizes 35 through 51; Europeans women's sizes 35 through 48.5; universal: small, medium, large, XL, XXL, and/or the like. Although specific shoe sizes have been recited herein, any size item of footwear is considered to falls within the scope of the present disclosure.

Moreover, in accordance with an exemplary embodiment, therapy shoe 100 is configured to be one-size-fits-all. For instance, portions of therapy shoe 100, such as coupling portion 102 and/or housing 101, may be expandable and/or retractable to accommodate a variety of foot sizes, widths, and lengths. In various exemplary embodiments, therapy shoe 100 is configured to be one of shaped to receive a right foot, shaped to receive a left foot, or shaped to receive a right or left foot. In certain exemplary embodiments, therapy shoe 100 may be provided in pairs, for example two therapy shoes 100, one each for the right foot and the left foot.

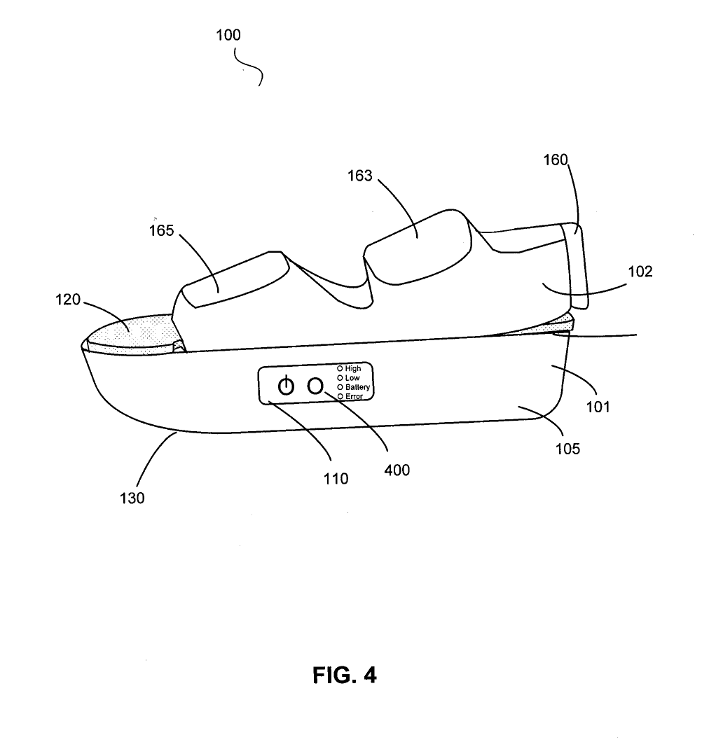

With reference now to FIG. 4, in accordance with an exemplary embodiment, a user (and/or caregiver, third person, etc.) may insert therapy device 400 into a cavity of therapy shoe 100 (not shown). Straps 160, 163, 165 (from a first closed position) of therapy shoe 100 are opened. A portion of a user, such as a lower extremity (e.g. foot), is inserted between a first and second side of straps 160, 163, 165, and comes into contact with surface 120. The location of the portion of a user with respect to surface 120 and/or other components of therapy shoe 120 may be a function of the location of therapy shoe 100's elements and/or a user's individualized anatomy. Once a portion of a user is in a desired position, straps 160, 163, 165 may be adjusted and/or closed (e.g., from a second position to a first closed position). Therapy device 400 may be activated through opening 110.

In various exemplary embodiments, therapy shoe 100 is configured to withstand force provided by a user while therapy shoe 100 is coupled to the user. For instance, a user may provide a force to therapy shoe 100 by standing or walking while coupled to therapy shoe 100. A user may move at any pace while coupled to therapy shoe 100. It will be appreciated that, in certain embodiments, therapy shoe 100 is configured to allow a user to remain mobile; however, in other exemplary embodiments, therapy shoe 100 is configured for use by a user who remains non-ambulatory while coupled to therapy shoe 100.

For example, in certain exemplary embodiments therapy shoe 100 is configured as a “walking” shoe. In these embodiments, therapy shoe 100 may be utilized by a wearer in a manner similar to a conventional shoe. In these embodiments, therapy device 400 may be configured to be inactive when the wearer is walking, for example in order to prevent wearer discomfort, damage to therapy device 400, and/or the like. In other exemplary embodiments, therapy shoe 100 is configured as a “non-walking” shoe. In these embodiments, therapy shoe 100 may be utilized by a wearer when non-ambulatory. In these embodiments, therapy device 400 may be configured to be constantly active, active at a scheduled interval, and/or the like.

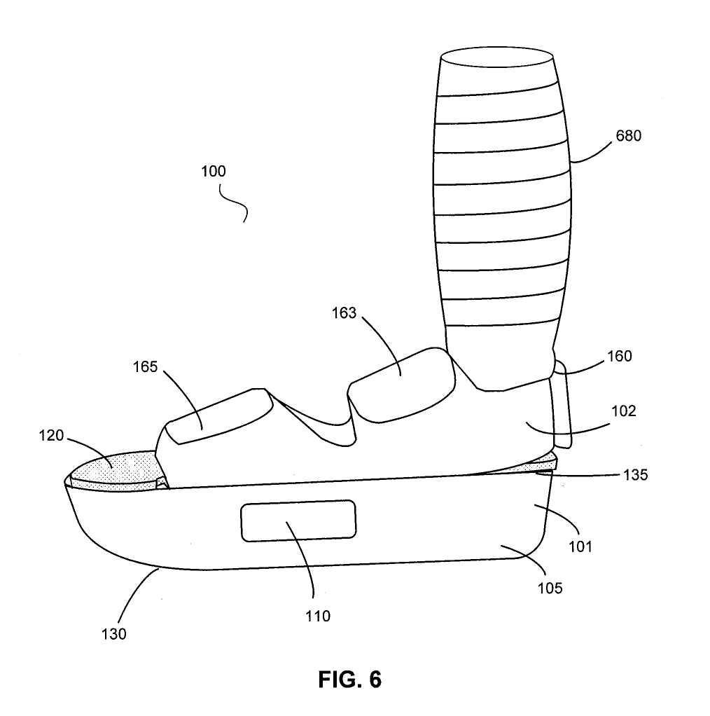

With reference now to FIG. 6, in various exemplary embodiments therapy shoe 100 may be coupled to and/or utilized in connection with a compression garment, for example compression sock 680. Compression sock 680 may be configured to work in a complementary manner with therapy device 400, for example in order to treat and/or prevent deep vein thrombosis, to facilitate athletic recovery, and/or the like. In an exemplary embodiment, compression sock 680 is releasably coupled to therapy shoe 100 via one or more of zippers, snaps, straps, buttons, hooks, hook and loop fasteners, and/or the like. In other exemplary embodiments, compression sock 680 is permanently coupled to therapy shoe 100, for example via gluing, stitching, and/or the like.

Compression sock 680 may comprise any suitable flexible material and may be configured with any suitable dimensions, shapes, curves, stitching, and/or the like, as desired, in order to at least partially receive and/or compress a portion of a limb. Moreover, compression sock 680 may be configured with any suitable level of compression, for example from between about 5 mmHg to about 50 mmHg. Compression sock 680 may be configured as knee-high, as thigh-high, as pantyhose, and/or in any other suitable configuration.

Because compression sock 680, when worn by a user, compresses the surface veins, arteries, muscles, and other soft tissue, circulating blood is forced through narrower circulatory channels, increasing the arterial pressure. This distribution of blood in the limb of a wearer can facilitate improved results (for example, increased blood circulation, decreased pooling of blood in the feet and lower extremities, etc.) responsive to operation of therapy device 400. Stated another way, when compression sock 680 and therapy device 400 work in tandem, blood flow is further concentrated and increased, with the result of facilitating athletic recovery, reducing deep vein thrombosis, reducing edema, and/or the like.

The present disclosure has been described above with reference to various exemplary embodiments. However, those skilled in the art will recognize that changes and modifications may be made to the exemplary embodiments without departing from the scope of the present disclosure. For example, the various operational steps, as well as the components for carrying out the operational steps, may be implemented in alternate ways depending upon the particular application or in consideration of any number of cost functions associated with the operation of the system, e.g., one or more of the steps may be deleted, modified, or combined with other steps. Further, it should be noted that while the methods and systems for aiding compression described above are suitable for use on the foot, similar approaches may be used on the hand, calf, extremities or other areas of the body. These and other changes or modifications are intended to be included within the scope of the present disclosure.

In the foregoing specification, the disclosure has been described with reference to various embodiments. However, one of ordinary skill in the art appreciates that various modifications and changes can be made without departing from the scope of the present disclosure as set forth in the claims below. Accordingly, the specification is to be regarded in an illustrative rather than a restrictive sense, and all such modifications are intended to be included within the scope of the present disclosure. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, required, or essential feature or element of any or all the claims. As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. Also, as used herein, the terms “coupled,” “coupling,” or any other variation thereof, are intended to cover a physical connection, an electrical connection, a magnetic connection, an optical connection, a communicative connection, a functional connection, and/or any other connection. Further, when language similar to “at least one of A, B, or C” is used in the claims, the phrase is intended to mean any of the following: (1) at least one of A; (2) at least one of B; (3) at least one of C; (4) at least one of A and at least one of B; (5) at least one of B and at least one of C; (6) at least one of A and at least one of C; or (7) at least one of A, at least one of B, and at least one of C.

What is claimed is:

1. A shoe, comprising:
a housing comprising a housing portion wherein the housing portion comprises;
a bottom surface,
a side surface attached to the bottom surface,
a top surface attached to the side surface wherein the top surface further comprises an aperture;
a housing cavity defined by the bottom surface, the side surface, the top surface;
wherein the housing cavity is configured to receive a therapy device.

2. The shoe of claim 1, further comprising a compression garment coupled to the housing.

3. The shoe of claim 1, wherein the aperture is configured with a moveable covering in order to conceal the control component of the foot compression system.

4. The shoe of claim 1, wherein the side surface has an area of between about 6 square centimeters to about 40 square centimeters.

5. The shoe of claim 1, wherein the shoe is an athletic shoe.

6. The shoe of claim 1 further comprising a therapy device disposed within the housing cavity wherein the therapy device is configured to operate when a wearer of the shoe is not walking and not operate when the wearer is walking.

7. A method for compressing a foot, the method comprising:
providing an item of footwear having a therapy device contained therein wherein the therapy device further comprises a power supply;
coupling the item of footwear to the foot; and
activating the therapy device.

8. The method of claim 7, wherein the activating the therapy device comprises repeatedly compressing the venous plexus region of the foot.

9. The method of claim 7, wherein the activating the therapy device comprises:
moving a pressure pad a first time to bring the pressure pad into contact with the foot to compress a portion of the foot;
moving the pressure pad a second time to bring the pressure pad out of contact with the foot to allow the portion of the foot to at least partially refill with blood; and
moving the pressure pad a third time to bring the pressure pad into contact with the foot to force at least a portion of the blood out of the portion of the foot.

10. The method of claim 7, further comprising coupling a compression garment to the limb associated with the foot.

11. The method of claim 7, wherein the therapy device is configured not to apply a compressive force to the foot when a user of the item of footwear is walking.

12. An item of footwear, comprising:
a housing having an aperture therethrough, the aperture configured with a flexible aperture cover;
a compression garment coupled to the housing;
a sole; and
a therapy device disposed between the housing and the sole, the therapy device configured to extend at least partially through the aperture to compress a portion of a foot;
wherein the item is footwear is configured to fully conceal the presence of the therapy device when the item of footwear is worn.

13. The item of footwear of claim 11, wherein at least one of the housing and the sole are disposable.

14. The item of footwear of claim 11, wherein the therapy device is entirely contained within the item of footwear.

15. An item of footwear, comprising:
a housing;
a therapy device disposed within a cavity in the housing; and
a coupling portion, the coupling portion configured to retain a portion of a foot.

16. The item of footwear of claim 15, wherein the housing further comprises an aperture disposed on a portion of the housing configured to be adjacent to a foot, wherein the aperture is configured to permit the therapy device to provide a compressive force to a portion of a foot

17. The item of footwear of claim 15, wherein the item of footwear comprises at least one of a boot, an athletic shoe, a dress shoe, or a sandal.

18. The item of footwear of claim 15, wherein the item of footwear is coupled to a compression garment.

19. The item of footwear of claim 15, wherein the aperture is at least partially covered by a flexible membrane.

20. The item of footwear of claim 15, wherein the item of footwear is similar in appearance to an item of footwear lacking a cavity configured to receive a therapy device.

21. The item of footwear of claim 15, wherein the item of footwear is configured to be wearable during exercise in order to facilitate athletic recovery.
